(19) United States
(12) Patent Application Publication
(10) Pub. No.: US 2023/0226750 A1
(43) Pub. Date: Jul. 20, 2023

(54) METHOD FOR PRODUCING A THREE-DIMENSIONAL OBJECT AND CORRESPONDING DEVICE

(71) Applicant: OTTOBOCK SE & CO. KGAA, Duderstadt (DE)

(72) Inventors: Olaf KROLL-ORYWAHL, Duderstadt (DE); Lars Benjamin FINKE, Duderstadt (DE); Leonard VIER, Duderstadt (DE)

(21) Appl. No.: 18/065,005

(22) Filed: Dec. 13, 2022

(30) Foreign Application Priority Data
Dec. 17, 2021 (DE) 10 2021 133 749.6

(51) Int. Cl. B29C 64/124; B29C 64/393; B29C 64/209; B29C 64/255

(52) U.S. Cl. CPC B29C 64/124; B29C 64/209; B29C 64/255; B29C 64/393; B33Y 10/00

(57) ABSTRACT

A method for producing a three-dimensional object by an additive manufacturing process includes introducing at least one manufacturing material fed in a flowable state from at least one feed-in opening of at least one feed-in needle into a supporting material. After being fed in, the at least one manufacturing material is cured, but it remains flexible or elastic after curing. The contour and/or position of at least one part of the three-dimensional object within the support material is detected by at least one sensor.

### FIELD OF THE INVENTION

The invention relates to a method for producing a three-dimensional object by means of an additive manufacturing process in which at least one manufacturing material is fed in a flowable state from at least one feed-in opening of at least one feed-in needle into a supporting material and then cures. The invention also relates to a device for conducting such a method, the device having a container that is filled or can be filled with support material and at least one feed-in needle, through which a manufacturing material can be introduced into the container.

### BACKGROUND

Nowadays, many types of additive manufacturing processes are known from the prior art and are used to produce a wide range of three-dimensional objects. Traditionally, additive manufacturing processes are hardly suitable for producing large quantities of the respective objects, as the production of individual objects takes a lot of time. In additive manufacturing processes, especially 3D printing, the object to be produced is built up of a number of very thin layers arranged on top of each other, which are often just a few millimetres thick. As a result, the production of large objects in particular is time-consuming and has therefore not yet become established for the production of large quantities.

MIT developed a three-dimensional printing method, which was published in US 2018/281295 A1 and is referred to as “Rapid Liquid Printing” (RLP). In this case, the object to be produced is made in a container that contains a gel suspension or another material as a support material which does not chemically react with the manufacturing material. It serves only to support the manufacturing material as long as it is not yet cured or sufficiently cross-linked. In the method, the manufacturing material is introduced in a flowable state, for example as a liquid or gel, at the desired positions within the support material. The feed-in needle is used for this purpose, which can be displaced in three directions that are linearly independent of one another. Due to the density ratios between the manufacturing material and the support material, the manufacturing material that has been introduced remains in the respective position, so that three-dimensional objects can be printed especially quickly by putting the manufacturing material at the desired points and subsequently cross-linking, setting or curing it there. This process is significantly faster than previous 3D printing methods and also makes it possible to produce flexible or elastic objects. It also allows a wide range of materials to be printed that have not been specifically optimized for additive manufacturing processes, but have become established in conventional casting processes. These include, for example, biocompatible silicones and especially elastomers with low Shore hardnesses for the production of elastic objects.

In comparison with conventional 3D printing processes, the disadvantage of RLP processes is that deformations still occur in the manufacturing material after being introduced into the support material. The pressure conditions change for the manufacturing material when the feed-in needle leaves through the at least one feed-in opening. As the manufacturing material is introduced in a flowable state, it can adapt to these different pressure conditions and deforms. In some manufacturing materials, especially those which are introduced in the form of multiple components, chemical reactions, such as cross-linking, occur after being introduced into the support material. This can also result in changes to flow properties or the volume of the material, which also leads to deformations in the manufacturing material that has been introduced.

The difficulty with RLP processes is therefore, among other things, knowing these flow properties and the resulting deformations of the manufacturing material as well as possible and taking them into account during production of the three-dimensional object. This is especially challenging with manufacturing materials that are elastic or flexible after curing. In this case, the three-dimensional object that is produced is also at least partially flexible or elastic, so that the contour changes upon removal of the object from the support material. Such a three-dimensional object may be the liner for a prosthesis, for example, that is designed to be elastic. If it or another elastic or flexible object is removed from the support material, gravity causes it to collapse, so that the produced and printed contour is no longer clear and therefore cannot be measured.

### SUMMARY

The invention is therefore based on the task of improving a method according to the preamble and eliminating or at least reducing the disadvantages of the prior art.

The invention solves the addressed task by way of a method characterized in that the contour and/or the position of at least one part of the three-dimensional object within the support material is detected by means of at least one sensor. Preferably, the contour of the entire three-dimensional object is detected by means of the at least one sensor.

The at least one sensor is therefore able to at least partially, but preferably completely, detect the contour, i.e. the geometric shape, of the three-dimensional object. This means that the sensor records sensor data from which conclusions can be drawn about the contour. It is not necessary for the sensor to directly detect the contour. The sensor data is preferably transmitted from the at least one sensor to an electrical control unit, especially an electronic data processing device, which is able to determine the contour from the sensor data by way of an algorithm executed by the control unit. The contour determined in this manner is the actual contour, which is then compared in the electrical control unit with a target contour, for example, which is preferably stored in an electronic memory that can be accessed by the electrical control unit. The result of the method and especially the quality of the three-dimensional object can thus already be identified in the support material, so that printing parameters, for example an exit rate of the manufacturing material through the feed-in opening of the feed-in needle, a speed at which the feed-in needle is moved through the support material, or the path along which the feed-in needle is moved, can be adapted and changed. The exact position of the three-dimensional object can also be detected in this way, so that further elements can be imprinted, for example. The position can change, especially during longer printing processes, if the object to be produced in the gel sags slightly due to the effect of gravity.

There is preferably at least one object in the support material whose contour and/or position within the support material is known or detected by means of the at least one sensor. Such an object is preferably already in the support material before the manufacturing material is introduced into the support material. The contour and/or position is preferably detected by the at least one sensor and saved. Such an object is, for example, an object that is to be joined to the manufacturing material or onto which the manufacturing material is to be printed. The manufacturing material is printed on when it is in contact with the object in a flowable and/or cured state. For example, the object may be a liner cap that is bonded with an elastic liner material which forms when the manufacturing material is in a flowable state.

Alternatively, the object can also be used to produce an outer surface and/or an inner surface of the three-dimensional object to be produced. If such a surface is to be especially smooth, for example, or feature a particular surface structure, the object can be used as a “mould”. The manufacturing material is printed onto the object in a free-flowing state and removed from the object after curing. The structure of the surface with which the object comes into contact with the manufacturing material is transferred to the respective surface of the three-dimensional object.

Preferably, the contour of the three-dimensional object comprises an outer surface and/or an inner surface and/or a wall thickness of the object. Particularly preferably, the contour of the three-dimensional object is formed by the entire surface of the object. It is especially preferable if this also includes the surface of cavities present in the object. Orthopaedic objects in particular, such as prosthesis liners, have an outer surface and an inner surface. A prosthesis liner is worn between an amputation stump and a prosthesis socket and acts as cushioning, for example. The prosthesis liner is usually made of an elastic material and is preferably individually adapted to the amputation stump and especially its contour. The prosthesis liner has an inner surface which, when mounted, faces the amputation stump of the wearer of the prosthesis system of which the prosthesis liner is a part. The inner surface comes into contact with the wearer’s skin. The prosthesis liner also has an outer surface which, when mounted, faces the prosthesis socket or at least faces away from the amputation stump. The contour preferably comprises both the outer surface and the inner surface of the prosthesis liner.

In a preferred embodiment, the at least one sensor features at least one optical sensor, such as a camera. In this case, the three-dimensional object is detected optically. The optical sensor can preferably detect visible light. However, it is also possible to use an optical sensor that can detect electromagnetic radiation outside of the optical spectral range, for example UV radiation or infrared radiation.

However, the support material is preferably transparent, or at least partially transparent, for electromagnetic radiation that can be detected by the optical sensor. In particular, in the event that the manufacturing material is not transparent for this electromagnetic radiation after curing, it makes sense to use an optical sensor. In a preferred embodiment, the three-dimensional object is illuminated with electromagnetic radiation, at least in the area whose contour is to be detected, which preferably corresponds to the electromagnetic radiation that can be detected by the optical sensor. Special lighting patterns can be used for this, for example parallel lighting strips of a preferably known width and at a preferably known distance from one another. The optical sensor then detects the parts of this lighting reflected by the three-dimensional object and the electrical control unit can draw conclusions about the contour of the three-dimensional object from the detected pattern. If the object is designed to be at least partially transparent for the electromagnetic radiation, the transmitter and the receiver can be arranged on opposite sides of the object. This means that the electromagnetic radiation is directed through the manufacturing material at least once on its way from transmitter to receiver. In this way, information about the object can be obtained from the absorption of the electromagnetic radiation. For example, methods known from tomography can be applied.

Alternatively or additionally, the at least one sensor has at least one ultrasonic sensor. In an especially preferred embodiment, the support material is an ultrasound gel that can also be used as an interface between the ultrasonic probe and the body of a wearer during an ultrasound examination. If the at least one sensor comprises at least one ultrasonic sensor, which is configured to detect ultrasonic waves, it is advantageous for the sensor itself or at least the device used to carry out such a method to also comprise an ultrasound source. Ultrasonic transmitters and ultrasonic receivers are usually arranged in a joint housing and together referred to as an ultrasonic probe. The ultrasonic transmitter and ultrasonic receiver can be the same ultrasonic transducer.

An advantage of using at least one ultrasonic sensor is that the ultrasonic waves are not only reflected on the outer surface of the object inside the support material, but also penetrate into the cured or curing manufacturing material and are also reflected at the nearest interface between two different materials, i.e. the inner surface of the three-dimensional object, for example. The ultrasonic waves received by the ultrasonic sensor and reflected at the interfaces between the different materials therefore contain information on both the outer surface and the inner surface of the three-dimensional object, so that the entire contour of the three-dimensional object can thus be detected.

Preferably, at least one sensor is moved relative to three-dimensional object in order to detect the contour. This is independent of the type of sensor and is particularly advantageous for optical sensors and ultrasonic sensors. The at least one sensor may be a mobile hand sensor, for example an optical scanner, or a moveable ultrasonic sensor, such as an ultrasonic probe. Alternatively or additionally, at least one sensor can be moved along a predetermined path. This path can be in the form of a rail or rail system, for example, and can lead at least partially, but preferably completely, around the container in which the support material is located and in which the three-dimensional object is printed. Alternatively, the sensor can be fixed to a movable arm, which is, for example, a robotic arm and can be driven by a motor and moved along a predetermined path controlled, for example, by an electrical control unit.

Advantageously, at least one sensor is positioned in the support material. The sensor is preferably fixed to a holder inside the container in which the support material is located. It preferably has an electrical energy storage device, such as a battery. The sensor advantageously has a wireless communication interface, for example a WiFi connection or a Bluetooth interface, with which the sensor is able to communicate wirelessly with an electrical control unit, in particular an electronic data processing device, and to forward measured values recorded by the sensor to this electrical control unit. Alternatively or additionally, power supply cables, for example electrical cables and/or data transmission cables, may be passed through the wall of the container in which the support material is located in order to supply the sensor located within the container with the required power and/or to enable communication. Preferably, the sensor positioned inside the support material is partially surrounded by the manufacturing material, so that it is especially easy for it detect an inner surface of the three-dimensional object.

The at least one sensor is preferably positioned in the support material in such a way that printing occurs at least partially around it and it can therefore detect the inner surface of the three-dimensional object. Preferably, the at least one sensor is already positioned in the support material before the manufacturing material starts to be introduced into the support material. A sensor arranged outside of the container in which the support material is located must also always penetrate the container wall and therefore more interfaces at which, for example, measurement radiation, such as electromagnetic radiation or ultrasound, can be reflected or refracted. A sensor positioned within the support material therefore has the advantage that there are fewer interfaces.

The contour is preferably detected after or during the curing of the manufacturing material.

Particularly preferably, at least one additional sensor, preferably an optical sensor, especially preferably a camera, is arranged on the feed-in needle, said sensor detecting the manufacturing material introduced through the feed-in needle. This means that not only is the contour of the three-dimensional object detected, but it is also possible to monitor the manufacturing material exiting from the feed-in opening of the feed-in needle. Information about, for example, the flow behavior of the manufacturing material can be gathered in this way. Such information can be consulted when determining printing parameters for further three-dimensional objects. It is also possible to monitor whether the manufacturing material is properly introduced into the support material. This concerns, for example, a bubble-free material flow.

Preferably, the detection of the contour and/or the position of the three-dimensional object is also performed during the printing process, i.e. while the manufacturing material is being introduced into the support material. In this way, the detected data, the information about the contour and/or position of the previously printed parts and/or exit properties of the manufacturing material from the feed-in needle can be transmitted to the electrical control unit. This enables a direct influence on the printing process and any errors that occur can be corrected quickly and easily. For example, printing could be stopped prematurely in the event of problems, or the specific parameter, such as wall thickness, could be readjusted by changing the printing parameters, such as the speed of the feed-in needle. Such a readjustment may be necessary, in particular, if properties of the support material and/or the manufacturing material deviate from expected properties or if other influencing factors change the printing result.

Constant monitoring of the printing process, especially with regards to the position of the three-dimensional object to be produced in the support material can increase the accuracy with which further objects are printed onto the three-dimensional object. This is especially important, as the objects may sink in the support material over the duration of the printing process. For example, if a sealing lip is to be printed onto a liner, it is important to do so at precisely the right height. The detection of further objects, which have preferably been introduced into the support material beforehand, may also be advantageous, for instance to enable printing onto them.

In addition, the invention solves the addressed task by way of a device for carrying out a method described which has a container that is filled or can be filled with support material and at least one feed-in needle, through which a manufacturing material can be introduced into the container. The device features at least one sensor, by means of which the contour of at least one part of the three-dimensional object located in the container can be detected. The device preferably has an electrical control unit, especially preferably an electronic data processing device, which is configured to process the sensor data detected by the at least one sensor. The sensor has a communication interface that interacts with a corresponding interface of the electrical control unit, so that the sensor transmits its data to the electrical control unit. The sensor data contains information about the contour of at least one part, but advantageously about the entire three-dimensional object. This contour is preferably extracted from the data in the electrical control unit and prepared for further processing. In a preferred embodiment, the device has an electronic memory in which data, for example target data for the contour of the object, are stored in electronic form. The electrical control unit can access the electronic memory and retrieve the target data and, for example, compare them with data on the contour of the object extracted from the sensor data, the so-called actual contour.

### DESCRIPTION OF THE DRAWINGS

In the following, a number of embodiment examples of the invention will be explained in more detail with the aid of the accompanying drawings. The numbers used in the drawings show the same elements.

FIG. 1 is a schematic representation of one embodiment of a device configuration for conducting a method for detecting a contour or position of an object in a container.

FIG. 2 is a schematic representation of another embodiment of device configuration for conducting a method for detecting a contour or position of an object in a container.

FIG. 3 is a schematic representation of a variation on the embodiment shown in FIG. 1 of a device configuration for conducting a method for detecting a contour or position of an object in a container.

FIG. 4 is a schematic representation of a variation on the embodiment shown in FIG. 2 of a device configuration for conducting a method for detecting a contour or position of an object in a container.

FIG. 5 is a schematic representation of yet another embodiment of device configuration for conducting a method for detecting a contour or position of an object in a container.

FIG. 6 is a schematic representation of a different variation on the embodiment shown in FIG. 1 of a device configuration for conducting a method for detecting a contour or position of an object in a container.

FIG. 7 is a schematic representation of a different variation on the embodiment shown in FIG. 2 of a device configuration for conducting a method for detecting a contour or position of an object in a container.

FIG. 8 is a schematic representation of still another embodiment of device configuration for conducting a method for detecting a contour or position of an object in a container.

FIG. 9 is a schematic representation of a further embodiment of device configuration for conducting a method for detecting a contour or position of an object in a container.

### DETAILED DESCRIPTION

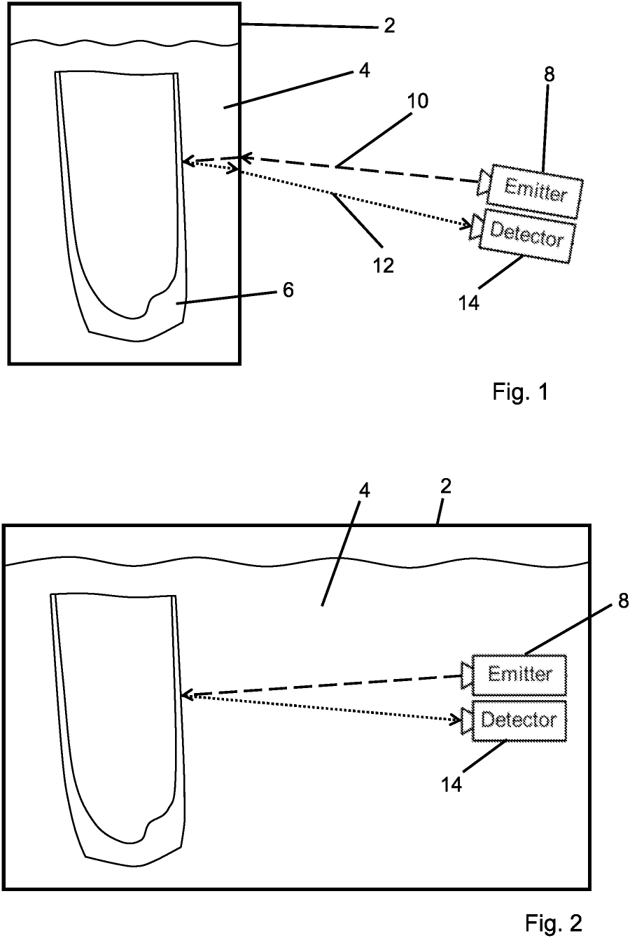

FIG. 1 depicts a container 2 in which support material 4 is located. A three-dimensional object 6 is depicted in the support material 4, wherein said object is to be measured and has been produced in the container 2. To this end an emitter 8 is shown, which emits measurement radiation 10. The measurement radiation 10 is refracted at the wall of the container 2 and strikes the object 6 in the support material 4. There, it is reflected and exits the container as reflected radiation 12. It is detected by a detector 14. The emitter 8 and the detector 14 are arranged outside of the container 2. In the embodiment example shown, they are not movable. Emitter 8 and detector 14 together form the detector that detects the contour and/or position of the object 6 within the container 2. The measurement radiation 10 can be strip lighting, for example.

FIG. 2 depicts a similar embodiment. The emitter 8 and the detector 14 are positioned within the container 2 and within the support material 4. This has the advantage that neither the measurement radiation 10 nor the reflected radiation 12 is refracted at the wall of the container 2.

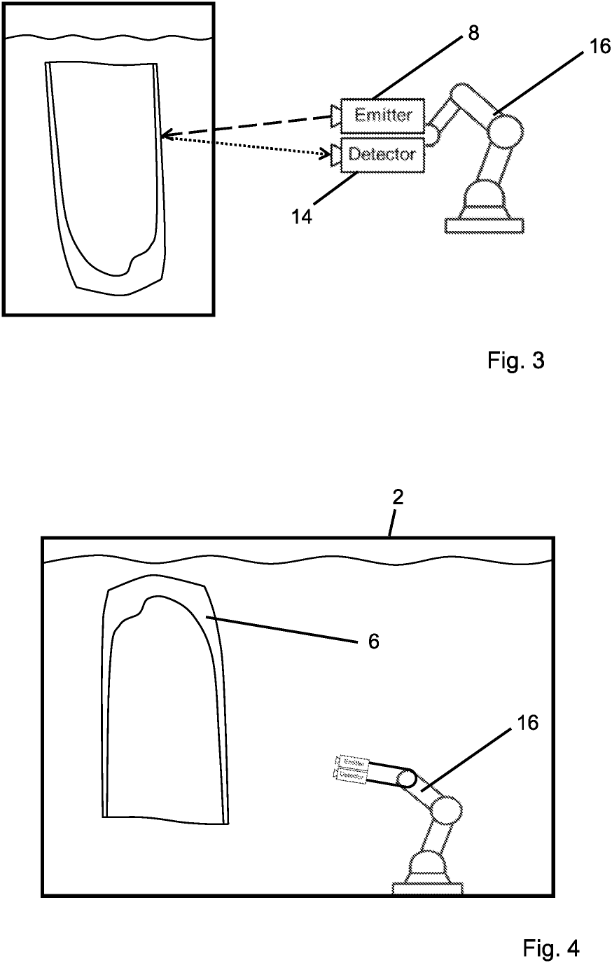

FIG. 3 shows an embodiment similar to FIG. 1. Here, too, the emitter 8 and the detector 14 are arranged outside of the container 2. Both are arranged together on an actuator 16, which in the embodiment example shown is designed as a robotic arm. As a result, emitter 8 and detector 14 can be moved collectively and it is possible to scan the object 6 and detect it from different sides and different perspectives. For the sake of simplicity, the measurement radiation 10 and the reflected radiation 12 are not shown as refracted at the wall of the container. Nevertheless, the radiation is refracted every time it passes through the interfaces.

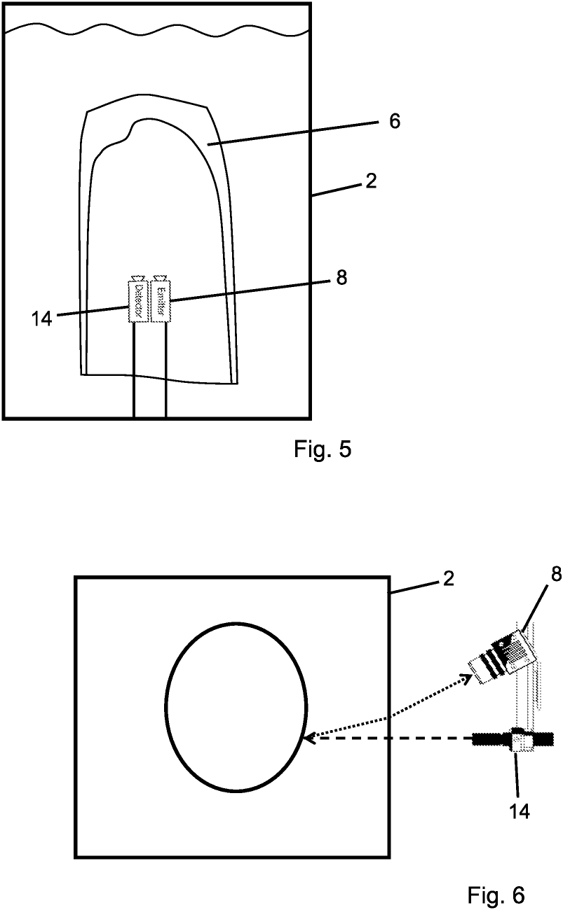

FIG. 4 shows the actuator 16 with emitter 8 and detector 14 within the container 2. As in the other figures, the object 6 is shown as a liner. In contrast to FIGS. 1 to 3, the liner in FIG. 4 has been produced with an open end 18 at the bottom, which is the proximal end. This makes it easier to remove the three-dimensional object 6 from the container 2.

In FIG. 5, the emitter 8 and the detector 14 are arranged within the object 6. Instead of detecting the outer side, as in the other figures, the embodiment in FIG. 5 detects the inner side of the object 6.

FIG. 6 corresponds to the representation from FIG. 1 with a different emitter 8 and a different detector 14. In this case, the emitter 8 is a laser, wherein the determination of the contour and/or position of the object in the embodiment example shown occurs via triangulation. Both are arranged outside of the container 2.

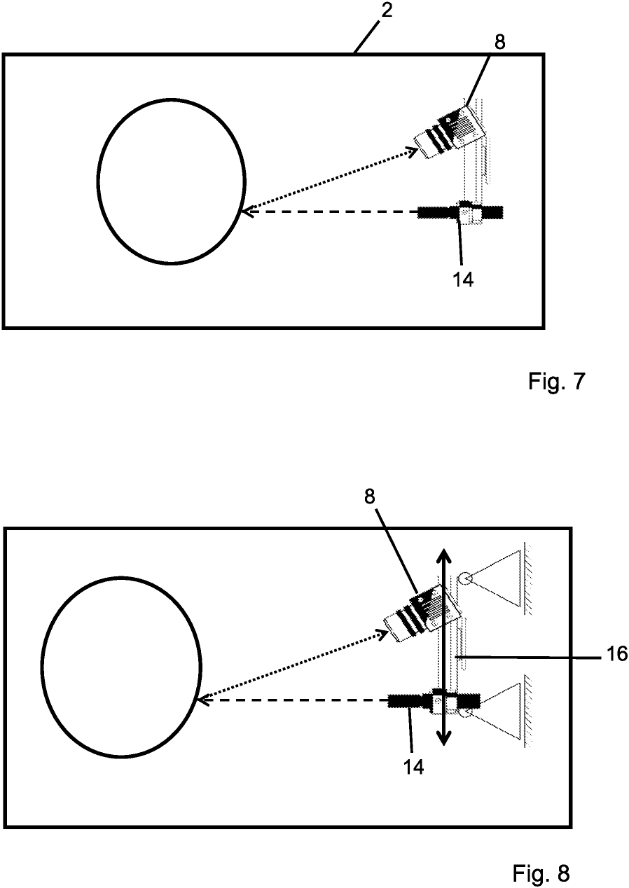

FIG. 7 depicts the emitter 8 and the detector 14 within the container 2.

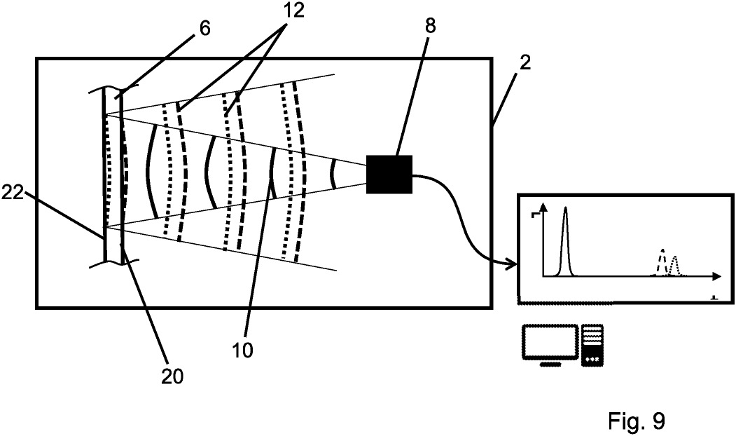

In FIG. 8, emitter 8 and detector 14 are arranged on a common actuator 16 by which they can be moved collectively.

FIG. 9 shows a different configuration. The emitter 8, which is designed as an ultrasonic transmitter, is inside the container 2. The measurement radiation 10 is emitted in the form of ultrasonic waves. It is represented by solid lines. The measurement radiation 10 strikes the object 6 and is reflected by the outer surface 20 and the inner surface 22. This results in two different reflected radiations 12, which are shown as dashed and dotted lines. The emitter 8 is simultaneously the detector 12 and sends the measurement data to an electrical control unit 24. The transmission peak 26 and the two reflection peaks 28 can be seen in the measurement data.

### Reference List

2 container
4 support material
6 object
8 emitter
10 measurement radiation
12 reflected radiation
14 detector
16 actuator
18 open end
20 outer surface
22 inner surface
24 electrical control unit
26 transmission peak
28 reflection peak

1. A method for producing a three-dimensional object by an additive manufacturing process, comprising:
introducing at least one manufacturing material in a flowable state from at least one feed-in opening of at least one feed-in needle into a supporting material;
curing the at least one manufacturing material to produce all or a portion of the three-dimensional object, wherein the at least one manufacturing material is elastic or flexible after curing; and
detecting at least one of a contour and a position of at least one part of the three-dimensional object within the support material with at least one sensor.

2. The method according to claim 1, wherein the detecting step detects the contour of an entirety of the three-dimensional object.

3. The method according to claim 1 further comprising having at least one object in the support material whose contour and/or position within the support material is known or which is detected by the at least one sensor.

4. The method according to claim 1 wherein the contour of the three-dimensional object detected by the at least one detector comprises one or more of an outer surface, an inner surface, and a wall thickness.

5. The method according claim 1 wherein the at least one sensor comprises at least one optical sensor.

6. The method according to claim 5 wherein the at least one sensor is a camera.

7. The method according claim 1 wherein the at least one sensor comprises at least one ultrasonic sensor.

8. The method according to claim 1 further comprising moving the at least one sensor relative to the three-dimensional object in order to detect the contour.

9. The method according to claim 1 wherein the at least one sensor is positioned in the support material.

10. The method according to claim 9, wherein the at least one sensor is positioned in the support material so as to detect an inner surface of the three-dimensional object.

11. The method according to claim 9 wherein the at least one sensor is positioned in the support material so as to permit printing to occur at least partially around the at least one sensor.

12. The method according to claim 1 wherein the contour is detected after or during the curing of the at least one manufacturing material.

13. The method according to claim 1 further comprising detecting the at least one manufacturing material introduced through the at least one feed-in needle using at least one additional sensor arranged on the at least one feed-in needle.

14. The method according to claim 1 wherein the step of detecting at least one of the contour and the position is performed while the at least one manufacturing material is introduced in the flowable state, and further comprising the step of influencing further introduction of the at least one manufacturing material based on the detected contour and/or position.

15. A device for conducting a method according to claim 1, comprising:
a container filled or fillable with support material;
at least one feed-in needle by which a manufacturing material can be introduced into the container; and
at least one sensor for detecting a contour of at least one part of a three-dimensional object located in or created by an additive process in the container.
